(19) United States
(12) Patent Application Publication
(10) Pub. No.: US 2020/0270578 A1
(43) Pub. Date: Aug. 27, 2020

(54) Method for Producing Mesenchymal Stem Cells from Neonatal Stem Cells

(71) Applicant: Joy Kong, Chatsworth, CA (US)

(72) Inventor: Joy Kong, Chatsworth, CA (US)

(21) Appl. No.: 16/286,482

(22) Filed: Feb. 26, 2019

(51) Int. Cl. C12N 5/0775

(52) U.S. Cl. CPC C12N 5/0662

(57) ABSTRACT

A method for producing mesenchymal stem cells from neonatal stem cells includes: extracting cells from an umbilical cord; extracting cells from an amniotic membrane, extracting cells from umbilical cord blood; producing serum from the umbilical cord blood; and suspending the cells in the serum of the umbilical cord blood; whereby about 20% to 30% of the total cells populations are mesenchymal stem cells. The method also includes cryopreserving the mesenchymal stem cells for storage at a temperature of about −80° Celsius. The method also includes performing the extractions from a C-section delivery. The method also includes requiring the donor answer a health questionnaire in regards to providing the umbilical cord, the amniotic membrane, and the umbilical cord blood.

### FIELD OF THE INVENTION

The present invention relates generally to a method for producing mesenchymal stem cells from neonatal birth tissue. More so, the present invention relates to a method for producing mesenchymal stem cells by extracting cells from the entire umbilical cord, which includes the umbilical cord tissue, the amniotic membrane, and remnants of umbilical cord blood; producing serum from the umbilical cord blood; and suspending the cells in the serum of the umbilical cord blood; whereby about 20% to 30% of the total cells populations are mesenchymal stem cells.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

Typically, Mesenchymal stem cells (“MSC” or “MSCs”) can be found in bone marrow, blood, dermis, periosteum and other tissues of the body, and are capable of differentiating into a variety of cell types, including adipose, areolar, osseous, cartilaginous, elastic, marrow stroma, muscle, fibrous connective tissue, and cardiac tissue, depending upon various in vivo or in vitro factors and influences. MSCs have been shown to engraft and selectively differentiate, based on the tissue environment, to lineages such as muscle, bone, cartilage, marrow stroma, tendon and fat. Due to their cellular origin and phenotype, these cells do not provoke an adverse immune response, allowing for the development of products derived from unrelated human donors.

In general, MSCs are isolated from the tissue from which they are obtained and purified. After isolation and purification, MSCs are subjected to a series of washes and, optionally, centrifugation. The MSCs then may be frozen and stored after the addition of an appropriate cryopreservative, such as glycerol (non-allergenic). Subsequently, the MSCs are thawed just prior to administration to a patient.

Those skilled in the art will recognize that umbilical cord tissue, such as Wharton's jelly, is rich in MSCs, the master of regeneration in the body. However, the MSCs from Wharton's jelly is more primitive than MSC's from the umbilical cord blood, and other prenatal tissue. Also, different components of the cord tissue possess MSCs that have different gene expression profile, surface markers, differentiation potentials, etc. Thus, the extraction of cells from the umbilical cord, amniotic membrane, and umbilical cord blood overcomes many of the limitations of prior art MSCs.

Other proposals have involved methods for extracting MSC from bone marrow, blood, dermis, periosteum and other tissues of the body. The problem is that most of these methods only produce less than 1% MSC. Also, they do not use young, neonatal MSC. MSCs derived from birth tissue have been shown to possess more potent proliferative potentials and wider range of differentiation potentials, with longer telomeres, senesce slower, and have higher anti-inflammatory properties. They also produce more growth factors to stimulate tissue repair, has less tumorigenic potentials and much stronger anti-cancer properties. Even though the above cited methods for extracting MSC's meet some of the needs of the market, a method for producing mesenchymal stem cells from neonatal stem cells by extracting cells from an umbilical cord; extracting cells from an amniotic membrane, extracting cells from umbilical cord blood; producing serum from the umbilical cord blood; and suspending the cells in the serum of the umbilical cord blood; whereby about 20% to 30% of the total cells populations are mesenchymal stem cells, is still desired.

### SUMMARY

Illustrative embodiments of the disclosure are generally directed to a method for producing mesenchymal stem cells from neonatal stem cells. The method involves the steps of: extracting cells from the entire umbilical cord, which includes the umbilical cord tissue, the amniotic membrane, and remnants of umbilical cord blood; producing serum from the umbilical cord blood; and suspending the cells in the serum of the umbilical cord blood; whereby about 20% to 30% of the total cells populations are mesenchymal stem cells.

The method is unique in that it performs cell extractions from the “entire cord” which includes the umbilical cord tissue and amniotic membrane, and may contain small amount of cord blood. The cells are then suspended in the serum of cord blood.

In another aspect, the method comprises a step of extracting the umbilical cord cells from an umbilical cord, and amniotic membrane, and umbilical cord blood from a C-section delivery.

In another aspect, the method comprises a step of providing a donor with a health questionnaire in regards to providing the umbilical cord, the amniotic membrane, and the umbilical cord blood.

In another aspect, the method comprises a step of providing a donor with an Informed Consent Form (GLP-DOC-0045).

In another aspect, the method comprises a step of providing a donor with a Donor Information and Health History Form (GLP-DOC-0046).

In another aspect, the method comprises a step of providing a donor with a Physical Evaluation Form (GLP-DOC-0047).

In another aspect, the method comprises a step of testing the umbilical cord blood for communicable diseases.

In another aspect, the mesenchymal stem cells from neonatal stem cells cryopreserved for storage.

In yet another aspect, the mesenchymal stem cells from neonatal stem cells are stored at a temperature range between −65° Celsius to 492° Celsius until use.

In yet another aspect, the mesenchymal stem cells from neonatal stem cells are stored at a temperature of −80° Celsius until use.

One objective of the present invention is to provide mesenchymal stem cells that are obtained from healthy newborn babies; and the associated tissues and cells.

Another objective is to produce mesenchymal stem cells from the cord tissue, cord blood, and amniotic membrane, allowing it to harness the regenerative potential of all three birth tissue compartments.

Another objective is to produce mesenchymal stem cells that are more primitive, metabolically more active, with faster rate of self-renewal, and wider range of differentiation.

Another objective is to secrete more growth factors, have longer telomeres, senesce (get old) slower.

Another objective is to produce mesenchymal stem cells that lack the accumulation of lifelong cellular damages of adult stem cells, which have gone through oxidative stress, radiation, or other toxic exposures associated with living and aging

Another objective is to produce mesenchymal stem cells that are highly rich in cytokines and growth factors, many of which are no longer present in adult tissue sources.

Another objective is to produce mesenchymal stem cells that have more anti-inflammatory and neuro-protective effects.

Yet another objective is to benefit from all the comprehensive nature of the stem cell composition by utilizing all of nature's intelligence, thus exerting the most profound benefits.

Other systems, devices, methods, features, and advantages will be or become apparent to one with skill in the art upon examination of the following drawings and detailed description. It is intended that all such additional systems, methods, features, and advantages be included within this description, be within the scope of the present disclosure, and be protected by the accompanying claims and drawings.

### BRIEF DESCRIPTION OF THE DRAWINGS

The invention will now be described, by way of example, with reference to the accompanying drawings, in which:

FIG. 1 illustrates a flowchart of an exemplary method for producing mesenchymal stem cells from neonatal stem cells, in accordance with an embodiment of the present invention;

FIG. 2 illustrates a prenatal diagram showing where the various prenatal cells are extracted from a region of the womb where the placenta is at the umbilical cord, in accordance with an embodiment of the present invention;

FIG. 3 illustrates a perspective view of a close up of the umbilical cord, in accordance with an embodiment of the present invention;

FIG. 4 illustrates a Table showing tissue source comparisons between placenta tissue, umbilical cord tissue, and autologous tissue, in accordance with an embodiment of the present invention; and

FIG. 5 illustrates a flowchart of an alternative embodiment of a method for producing mesenchymal stem cells from neonatal stem cells, in accordance with an embodiment of the present invention.

Like reference numerals refer to like parts throughout the various views of the drawings.

### DETAILED DESCRIPTION OF THE INVENTION

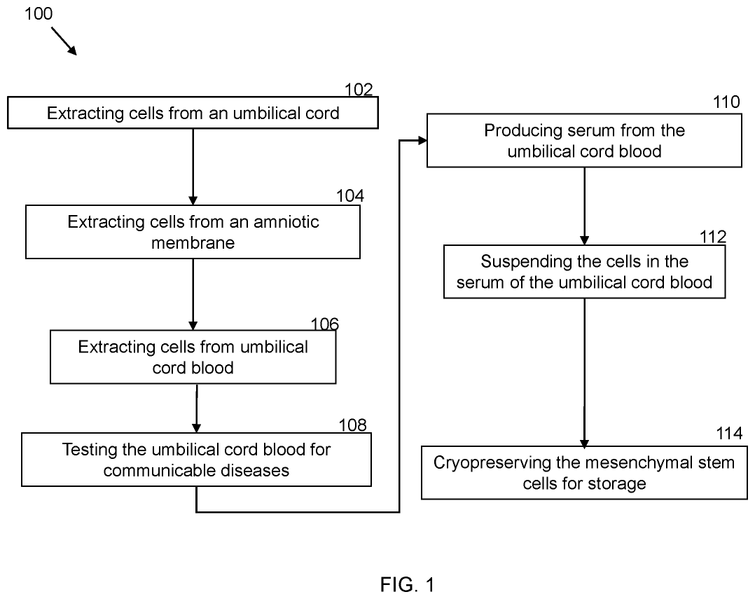

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper,” “lower,” “left,” “rear,” “right,” “front,” “vertical,” “horizontal,” and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Specific dimensions and other physical characteristics relating to the embodiments disclosed herein are therefore not to be considered as limiting, unless the claims expressly state otherwise.

A method for producing mesenchymal stem cells from neonatal stem cells is referenced in FIGS. 1-5. The use of neonatal cells, rather than older bone marrow or other known sources of mesenchymal stem cells, creates numerous advantages. None of which is greater than the fact that about 20% to 30% of the total cell populations produced are mesenchymal stem cells (MSC). This is larger than the 1% associated with other MSC extraction means. Thus, the MSC is processed from the cord tissue, cord blood and amniotic membrane, allowing it to harness the regenerative potential of all three birth tissue compartments.

In essence, the method includes: extracting cells from an umbilical cord; extracting cells from an amniotic membrane, extracting cells from umbilical cord blood; producing serum from the umbilical cord blood; and suspending the cells in the serum of the umbilical cord blood; whereby about 20% to 30% of the total cells populations are MSC. The method also includes cryopreserving the mesenchymal stem cells for storage at a temperature of about −80⁰ Celsius. The method also includes performing the extractions from a C-section delivery. The method also includes providing a donor with a health questionnaire in regards to providing the umbilical cord, the amniotic membrane, and the umbilical cord blood.

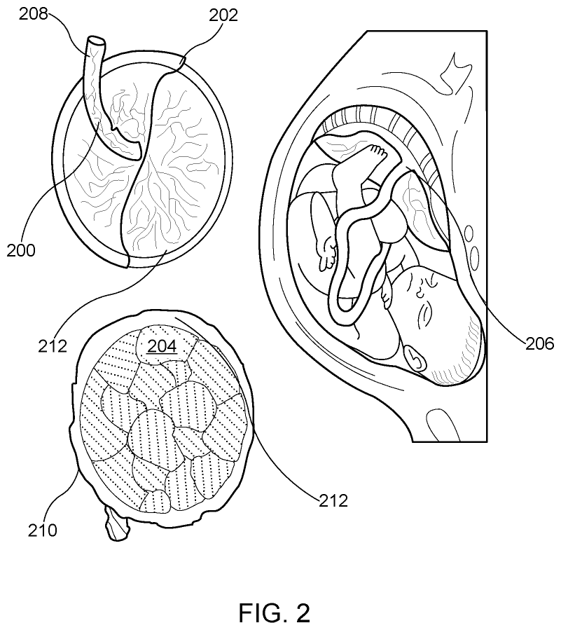

As referenced in the flowchart of FIG. 1, the method produces MSC from the cord tissue, cord blood and amniotic membrane, allowing it to harness the regenerative potential of all three birth tissue compartments. FIG. 2 illustrates a prenatal diagram showing how the various prenatal cells are extracted from a region of the womb 206 where the placenta is at the umbilical cord. It is significant to note that the method performs the extractions only from cells obtained from a C-section delivery; not a standard vaginal delivery.

Looking again at the flowchart of FIG. 1, an initial Step 102 comprises extracting cells from an umbilical cord. As is known in the art, the umbilical cord 200 is a conduit between the developing embryo or fetus and the placenta. The umbilical cord 200 is shown in FIG. 2 extending through an amniotic membrane 202. The umbilical cord 200 has a fetal surface 208 from which cells may be extracted. The method may further comprise a Step 104 of extracting cells from an amniotic membrane. As those skilled in the art will recognize, the amniotic membrane 202 is the innermost layer of the placenta consisting of a thick basement membrane and an avascular stromal matrix. Extracting cells therefrom is one of the vital steps of the method. Guidance for extraction of cells, as applied to the present invention is found in Appendix C and Appendix D.

A Step 106 includes extracting cells from umbilical cord blood. The umbilical cord blood 204 is contained in the umbilical cord 200, and is carried through a chorion 212. By penetrating the maternal surface 210 of the umbilical cord 200, the umbilical cord blood 204 may be extracted. Looking at prior art mesenchymal stem cell extraction means, the tissue-derived stem cell products are either cord blood based (containing less than 0.1% MSC's), or cord tissue-based (containing between 20% to 100% MSC's). But there is no mesenchymal stem cell that contains both, plus the amniotic membrane, plus the serum of the cord blood.

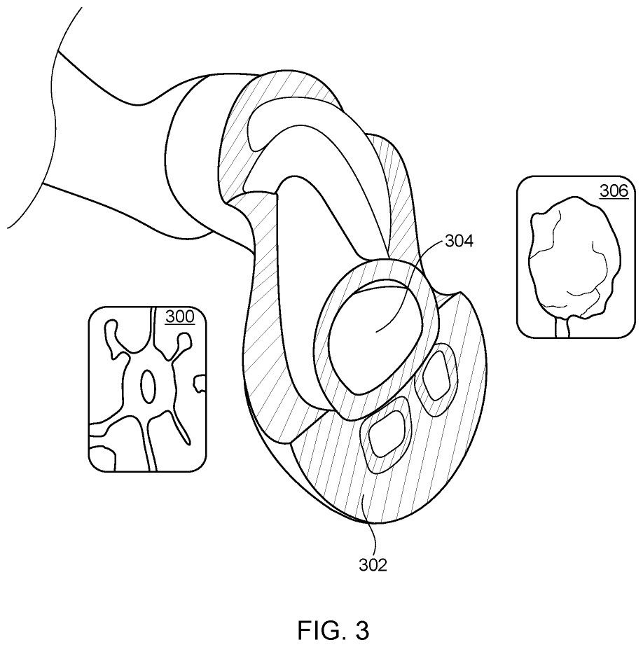

Such a combination offered by the present method is illustrated in a close up of the umbilical cord, shown in FIG. 3, allows for extraction of a higher proportion (20-30%) of MSC 300, and also hematopoietic stem cells 306 that the cord blood 304 and cord tissue 302 contains; as well as various immature immune cells, endothelial progenitor cells that are part of the MSC's. Further, the method allows for the production of beneficial cytokines/growth factors and proteins such as TIMP-2 protein, which is at high amount in cord blood serum.

In one non-limiting embodiment of the neonatal MSC produced by the method, 8-10 million cells/cc are produced, with components from the entire umbilical cord, including umbilical cord tissue, Wharton's jelly, amniotic membrane and cord blood. The neonatal MSC contains rich content of MSC's, some HSC's (Hematopoietic Stem Cells), and numerous growth factors and cytokines. Further, the neonatal MSC's can be used in any form of injections s they are safe for IV use. (See Also Appendix B)

Those skilled in the art will recognize that umbilical cord tissue, such as Wharton's jelly, is rich in MSCs, the master of regeneration in the body. However, the MSCs from Wharton's jelly is more primitive than MSC's from the umbilical cord blood, and other prenatal tissue. Also, different components of the cord tissue possess MSCs that have different gene expression profile, surface markers, differentiation potentials, etc. Thus, the extraction of cells from the umbilical cord, amniotic membrane, and umbilical cord blood overcomes many of the limitations of prior art MSCs.

In some embodiments, a Step 108 comprises testing the umbilical cord blood for communicable diseases. Once the donor tissue is accepted, the donor's blood is tested for relevant communicable diseases in a laboratory certified under Clinical Laboratory. In one embodiment, the umbilical cord blood is screened for communicative disease according to AATB (American Association of Tissue Banks) standards. Donor screening, tissue collection and processing protocols meet or exceed applicable FDA regulations and industry standards.

A licensed physician must review the results of testing and determine that the donor has met all eligibility requirements. The physician utilizes available relevant information which may have included, but not limited to: donor interview, medical/hospital records, donor physical assessment, infectious disease test results, radiology/pathology and other records if available and pertinent. Recipient records must be maintained for the purpose of tracing tissue post-transplant per JCAHO and FDA requirements.

A Step 110 includes producing serum from the umbilical cord blood. The serum from cord blood is carefully added back to the final product. The umbilical blood serum contains valuable proteins/growth factors, such as TIMP-2 protein, which has been shown to promote memory and learning. The serum may be produced using known serum-producing means. In one non-limiting embodiment, the serum extraction includes: drawing X mL of whole blood for each X mL of serum or plasma needed; collecting in an appropriate collection tube; centrifuging the blood for at least 15 minutes at 2200-2500 RPM; and administrating, with a pipette, the serum into a clean screw-cap vial. However other serum extraction means may be used.

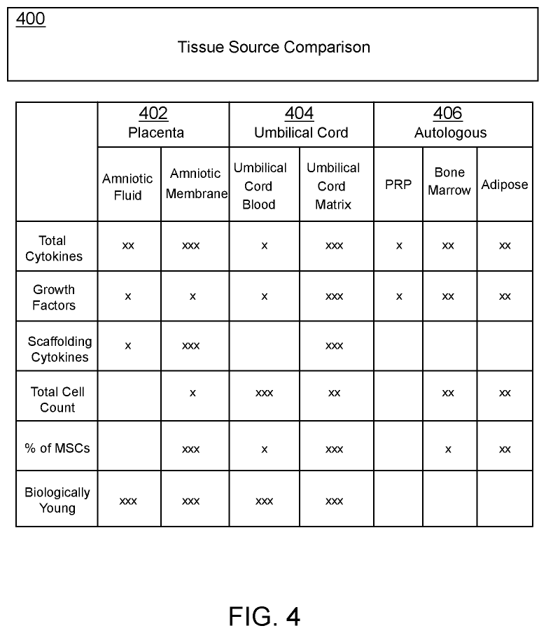

In some embodiments, a Step 112 may include suspending the cells in the serum of the umbilical cord blood. During the suspension, the MSC cell population is produced for harvesting. At this suspension stage, about 20% to 30% of the total cells populations produced are MSC. The neonatal cells create such large proportions of MSC. This is further illustrated in FIG. 4, which references Table 400, showing tissue source comparisons between placenta tissue 402, umbilical cord tissue 404, and autologous tissue 406. The placenta and umbilical cord tissues 402, 404 are at issue with the present invention. And it is known in the art that autologous stem-cell transplantation is autologous transplantation of stem cells—that is, transplantation in which stem cells are removed from a person, stored, and later given back to that same person

Continuing with Table 400, the total cytokine count is about equivalent for the three tissues 402, 404, 406. However growth factors are greatest in the umbilical cord tissue 404. Growth factors are greatest with autologous tissue 406. Scaffolding cytokines are greatest with the placenta tissue 402. However, the important Total Cell Count is greatest with the umbilical cord tissue 404. Further, the percentage of MSC is greatest with the umbilical cord tissue 404. Both the placenta tissue 402 and the umbilical cord tissue 404 are classified as biologically young, which helps explain why the MSC percentage is greater than for the older autologous tissue 406. (See Also Appendix A)

Aside from the produced MSC's, the method produces other mononuclear cells, including hematopoietic progenitor cells, endothelial progenitor cells, and immature immune cells, etc. The mononuclear cells contain a multitude of multipotent progenitor cells that can differentiate into blood cells, endothelial cells, hepatocytes, myocytes, cardiomyocytes, smooth muscle cells, epithelial cells, neural cells, osteoblasts, fibroblasts, etc. For example, In lcc of the produced MSC (8 million cells per cc on average), it would contain 1.6 to 2.4 million MSC's, which is at least twice the number of MSC's compared to the best performing competitor product that is extracted from cord tissue.

A Step 114 comprises cryopreserving the mesenchymal stem cells for storage. Those skilled in the art will recognize that cryopreserving is a process where the cells and tissues, which are susceptible to damage caused by unregulated chemical kinetics, are preserved by cooling to very low temperatures. In one embodiment of the method, the storage may be at a temperature range between −65° Celsius to −192° Celsius. However in other embodiments, the mesenchymal stem cells can be stored at a temperature of about −80° Celsius.

In revision, a possible first step of the method may include providing a donor with a health questionnaire. The questionnaire can be used to form information and an opinion about the donor's umbilical cord, amniotic membrane, and umbilical cord blood. This helps ensure that the cells and blood are pure. Other forms that may require filling out by the donor may include, without limitation, an Informed Consent Form (GLP-DOC-0045), a Donor Information and Health History Form (GLP-DOC-0046), and a Physical Evaluation Form (GLP-DOC-0047).

The Informed Consent Form (GLP-DOC-0045) is completed and signed by the Donor Medical records do not identify areas that would cause the donor to be ineligible per the criteria identified in the FDA Guidance Document Eligibility Determination for Donors of HCT/Ps (August 2007) or within the ineligibility criteria in this procedure. The Donor Information and Health History Form (GLP-DOC-0046) does not identify areas that would make the donor ineligible. (See Also Appendix B)

Further, the communicable disease testing is performed by a CLIA certified lab and includes negative results for required testing. The Physical Evaluation Form (GLP-DOC-0047) does not identify physical evidence of sexually transmitted diseases, syphilis, non-medical percutaneous drug use such as needle tracks, disseminated lymphadenopathy, unexplained oral thrush, jaundice, sepsis, large scabs, severely necrotic lesions, etc. The recovery location of the donated FICT/P was suitable as indicated on the Postnatal Birth Tissue Recovery Form (UP-DOC-0048).

Donors are screened and selected based on stringent criteria. Potential donating mothers are examined periodically by an obstetrician throughout pregnancy, and are required to go through necessary testing. Before time of birth, if a mother chooses not to store their baby's umbilical cord for private use, she is asked if she would like to donate birth tissue (placenta and umbilical cord). When their answer is yes, the mother is asked to complete an extensive questionnaires, which screen for infectious disease exposures, alcohol/drug/smoking or other toxic exposures, medication use, travel history, any family history of heritable and non-heritable diseases, such as cancer, autoirnmune diseases, blood disorder, etc., partner's health history (baby's father), as well as any prenatal test abnormality of the baby. Informed consent is obtained, and the final acceptance of the donor tissue is determined by a licensed physician.

In use, the neonatal MSC is injectable and pre-mixed in a flowable birth tissue matrix, formulated specifically for in-office use. These allograft products contain cellular material, collagens, growth factors, and other key biologic components. They are all intended for homologous use for the repair, replacement, reconstruction, or augmentation of human tissue.

In an exemplary Foundation for Regeneration, the birth tissue ECM contains Collagens I, Ill, IV, V, VI, VII, fibrous proteins that provide a structural scaffold to support cellular migration. Fibronectin, integrins, laminins, and hyaluronons also play a key role in proliferation, differentiation and adherence to the scaffold.

In an exemplary Modulate Correct Tissue Repair, growth factors contained in the birth tissue ECM, including PDGF, VEGF, EGF, FGF and TGF-B, support cell proliferation and migration across the defect. This combination of proteins works with the body's own cells to modulate correct tissue reconstruction rather than scar tissue. Cellular components, especially MSC's, further deliver targeted signals to promote tissue repair.

In an exemplary Regulation of Inflammation, Scarring and Pain, birth tissue products have been shown to reduce inflammation, fibrous tissue growth, and potential scar tissue formation. In an exemplary Non-Immunogenic Birth, tissue-derived stem cell products are ‘immune-privileged’, possessing little or no risk of foreign body reaction, which can lead to fibrosis and graft failure. In an exemplary Anti-Microbial Application, birth tissue products has been shown to reduce bacteria counts in the wound, demonstrated against a wide range of bacteria.

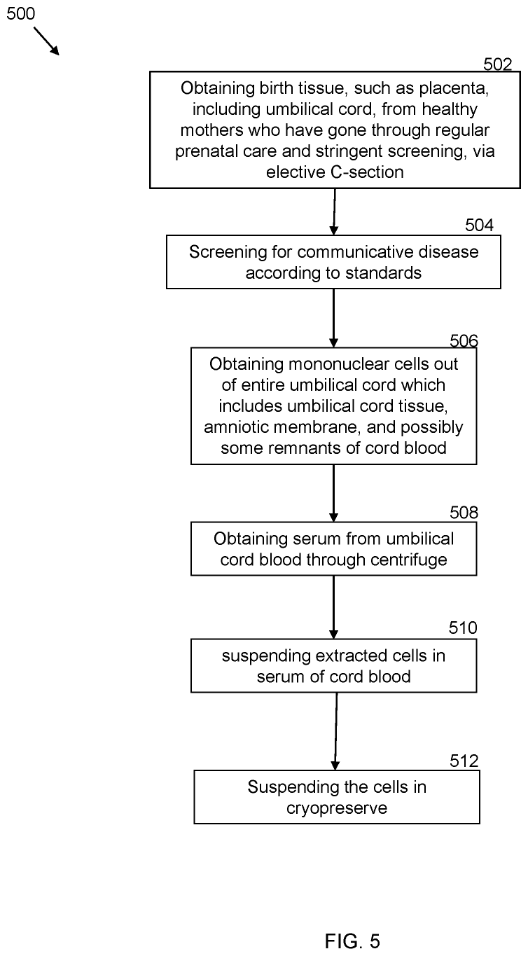

A variation of the present invention is found in FIG. 5, which references a similar method 500 for producing mesenchymal stem cells from neonatal stem cells. Method 500 includes a first Step 502 of obtaining birth tissue, such as placenta, including umbilical cord, from healthy mothers who have gone through regular prenatal care and stringent screening, via elective C-section. A subsequent Step 504 of screening for communicative disease according to standards.

Another Step 506 may include obtaining mononuclear cells out of entire umbilical cord which includes umbilical cord tissue, amniotic membrane, and possibly some remnants of cord blood. Another Step 508 includes obtaining serum from umbilical cord blood through centrifuge. A step 510 includes suspending extracted cells in serum of cord blood. Another Step 512 comprises suspending the cells in cryopreserve.

Although the process-flow diagrams show a specific order of executing the process steps, the order of executing the steps may be changed relative to the order shown in certain embodiments. Also, two or more blocks shown in succession may be executed concurrently or with partial concurrence in some embodiments. Certain steps may also be omitted from the process-flow diagrams for the sake of brevity. In some embodiments, some or all the process steps shown in the process-flow diagrams can be combined into a single process. It is significant to note that all the cells used in the method (derived from cord blood, cord tissue, and amniotic membrane) are from a single donor within each vial and each lot. In one non-limiting embodiment, each vial has lot number and donor ID number that can be traced to the origin.

These and other advantages of the invention will be further understood and appreciated by those skilled in the art by reference to the following written specification, claims and appended drawings.

Because many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalence.

What is claimed is:

1. A method for producing mesenchymal stem cells from neonatal stem cells, the method comprising:
obtaining birth tissue, such as placenta, including umbilical cord, from healthy mothers who have gone through regular prenatal care and stringent screening, via elective C-section;
screening for communicative disease according to standards;
obtaining mononuclear cells out of entire umbilical cord which includes umbilical cord tissue, amniotic membrane, and possibly some remnants of cord blood;
obtaining serum from umbilical cord blood through centrifuge;
suspending extracted cells in serum of cord blood; and
suspending the cells in cryopreserve.

2. The method of claim 1, wherein the cells are stored at a temperature range between −65° Celsius to −192° Celsius.

3. The method of claim 1, wherein the standards comprise AATB standards.

4. A method for producing mesenchymal stem cells from neonatal stem cells, the method comprising:
extracting cells from an umbilical cord;
extracting cells from an amniotic membrane;
extracting cells from umbilical cord blood;
producing serum from the umbilical cord blood; and
suspending the cells in the serum of the umbilical cord blood.

5. The method of claim 4, wherein about 20% to 30% of the total cells populations are mesenchymal stem cells.

6. The method of claim 4, further comprising a step of extracting the umbilical cord cells from an umbilical cord, and amniotic membrane, and umbilical cord blood from a C-section delivery.

7. The method of claim 4, further comprising a step of providing a donor with a health questionnaire in regards to providing the umbilical cord, the amniotic membrane, and the umbilical cord blood.

8. The method of claim 7, further comprising a step of providing the donor with an Informed Consent Form (GLP-DOC-0045).

9. The method of claim 8, further comprising a step of providing the donor with a Donor Information and Health History Form (GLP-DOC-0046).

10. The method of claim 9, further comprising a step of providing the donor with a Physical Evaluation Form (GLP-DOC-0047).

11. The method of claim 4, further comprising a step of testing the umbilical cord blood for communicable diseases.

12. The method of claim 4, further comprising a step of cryopreserving the mesenchymal stem cells for storage.

13. The method of claim 12, wherein the mesenchymal stem cells are stored at a temperature range between −65° Celsius to −192° Celsius.

14. The method of claim 12, wherein the mesenchymal stem cells are stored at a temperature of about −80° Celsius.

15. A method for producing mesenchymal stem cells from neonatal stem cells, the method comprising:
extracting cells from an umbilical cord;
extracting cells from an amniotic membrane;
extracting cells from umbilical cord blood,
whereby the extractions are from a C-section delivery;
testing the umbilical cord blood for communicable diseases;
producing serum from the umbilical cord blood;
suspending the cells in the serum of the umbilical cord blood,
whereby about 20% to 30% of the total cells populations are mesenchymal stem cells; and
cryopreserving the mesenchymal stem cells for storage.

16. The method of claim 15, further comprising a step of providing a donor with a health questionnaire in regards to providing the umbilical cord, the amniotic membrane, and the umbilical cord blood.

17. The method of claim 16, further comprising a step of providing the donor with an Informed Consent Form (GLP-DOC-0045).

18. The method of claim 17, further comprising a step of providing the donor with a Donor Information and Health History Form (GLP-DOC-0046).

19. The method of claim 15, wherein the mesenchymal stem cells are stored at a temperature range between −65° Celsius to 492° Celsius.

20. The method of claim 15, wherein the mesenchymal stem cells are stored at a temperature of about −80° Celsius.
